(19) United States
(12) Patent Application Publication
(10) Pub. No.: US 2012/0021477 A1
(43) Pub. Date: Jan. 26, 2012

(54) Method for the Fixation of CO2 and for Treating Organic Waste by Coupling an Anaerobic Digestion System and a Phytoplankton Microorganism Production System

(76) Inventors: Olivier Bernard, Carros (FR); Gael Bougaran, Nantes (FR); Jean-Paul Cadoret, Basse Goulaine (FR); Raymond Kaas, Chapelle Sur Erdre (FR); Eric Latrille, Salles D'aude (FR); Bruno Sialve, Narbonne (FR); Jean-Philippe Steyer, Nevian (FR)

(21) Appl. No.: 13/144,640

(22) PCT Filed: Jan. 5, 2010
(86) PCT No.: PCT/FR10/50008

§ 371 Date: Sep. 30, 2011

(30) Foreign Application Priority Data
Jan. 20, 2009 (FR) 0950334

(51) Int. Cl. C12P 5/02; C12M 1/12; C12M 1/107

(52) U.S. Cl. 435/167; 435/300.1; 435/297.1

(57) ABSTRACT

The invention relates to a CO₂ fixation and organic waste processing method, wherein microorganisms (105) from a phytoplanktonic culture and organic waste (104) are processed inside a hydrolysis reactor (101); at least part of a liquid effluent (109) exiting the hydrolysis reactor is processed inside a methanation reactor (102); a liquid phase (127) and biogas to be purified (110) exiting Step (a″) are processed inside a phytoplanktonic microorganism culture unit (103); a CO₂-containing gaseous effluent (113) is injected into the phytoplanktonic microorganism culture unit; an NH₃ concentration under 0.5 g/L is maintained inside the methanation reactor; and a methane-enriched biogas is recovered upon exiting the phytoplanktonic microorganism culture unit. The invention also relates to a system for implementing this method.

CO₂ fixation and organic waste treatment method combining an anaerobic digestion system with a system for producing phytoplanktonic microorganisms.

### FIELD OF THE INVENTION

The invention relates to a combined CO₂ fixation and organic waste processing method for processing various types of organic waste and for simultaneously capturing large quantities of CO₂, which are harmful to the environment and originate from gaseous industrial waste effluents, while producing methane-rich purified biogas. More specifically, the invention relates to a method that, by recycling nitrogen and other nutrients originating from anaerobic digestion of organic waste, makes it possible to process the high-volume CO₂ flows involved therein by using phytoplanktonic microorganisms such as microalgae and/or photosynthetic bacteria. The method of the invention therefore enables fixation of CO₂ that is normally released into the atmosphere, which contributes to the greenhouse effect, and transforming it into bioenergy.

The invention can be applied to any industry that generates organic waste, particularly to the food industry and to CO₂-emitting human activities. Applications for the invention also exist in the field of biofuel production, since the purified biogases resulting from the method of the invention are methane-enriched, and since the biomass (specifically, the algal biomass) used for biogas purification is a rich source of lipids that can also be used as biofuels.

### STATE OF THE ART

Methanation has long been used for transforming soluble or solid organic waste into biogas. This technique is applied to remediation of pollution loads such as urban or industrial wastewater containing biodegradable organic matter or organic products in solid form, such as sewage sludge, household waste, biowaste, food industry waste, waste from various agricultural or forestry-related activities, and energy crops. Converting organic matter into methane by biological means offers the advantage of providing energy that can be used directly as fuel in vehicles, or that can be converted into heat and/or electricity.

Processing solid loads via methanation, when performed in a single step, is quite time-consuming. For this reason, a two-step method is known: the first step involves hydrolysis, wherein macromolecules are transformed into low-molecular-weight, soluble molecules, and acidogenesis, wherein the low-molecular-weight molecules are transformed into short-chain organic acids, alcohols, hydrogen, and other simple compounds; and, in the second step, acetogenesis, wherein the alcohols and organic acids are transformed into acetic acid, and methanogenesis, wherein methane is formed either from hydrogen or acetic acid.

The biogas produced during methanation is primarily composed of a 50 to 70% mixture of methane and 30 to 50% carbon dioxide. However, depending upon the reactor's operating conditions, but also upon the nature of the substrate(s), other compounds (hydrogen sulfide, ammonia, siloxanes, etc.) may be present in various concentrations. Even in trace amounts in biogas, these substances may harm downstream energy transformation processes (corrosion of mechanical parts, fouling of engines/turbines, etc.).

Therefore, in most cases, conversion into mechanical or electrical energy requires a prior filtration/purification step for sustainable production.

At present, numerous research efforts are focusing on mass-producing microalgae to be used in energy. As is the case for higher plants, microalgae need a carbon source (inorganic—CO₂ or HCO₃⁻—or organic—acetate, glucose, etc.) in order to grow, along with nutrients (nitrogen, phosphorus, etc., but also trace elements, occasionally vitamins, etc.) and light energy. Under nutritional stress conditions, certain microalgae species are able to accumulate a large quantity of carbon in lipid form. The lipid production capacity of certain species is considerably higher than that observed for land-based oleaginous species, which makes microalgae especially attractive for biofuel systems.

However, large-scale production of these microorganisms involves mobilizing a very large quantity of nutrients. According to the Redfield ratio (C/N/P:106/16/1), for one ton of fixated CO₂ (which corresponds to 600 kg of produced dried biomass), approximately 50 kg of nitrogen and 3.1 kg of phosphorus must be mobilized. Bearing in mind that, for an open pond with a one-hectare surface area, it is possible to fixate one ton of CO₂ per day, the required amounts of nitrogen and phosphorus are high and exceed the needs of land-based oleaginous crops.

Moreover, the carbon source—in the form of CO₂—is the limiting factor for microalgae growth. A continuous supply of CO₂ during the light phase of photosynthesis yields high productivity levels in terms of biomass and also encourages (under certain conditions) higher lipid accumulation. However, adding CO₂ to the culture medium results in a drop in pH that may be harmful for certain species. This toxicity caused by excess acidity requires that the CO₂ flow be precisely controlled based on the medium's pH. Therefore, it is advisable to link the injection of CO₂-rich gas into the medium with the pH such that the pH is kept at a set point.

Growing [microalgae] inside closed systems (photobioreactors) results in very high productivity levels in terms of CO₂ fixation and biomass production. However, the costs associated with technology of this type (construction, operation, maintenance) make this technology largely incompatible with mass production, at least for the time being. Conversely, open systems, which are much more attractive in terms of cost, are extremely sensitive to various types of contamination (native microalgae, bacteria, predators).

Additionally, operations for harvesting microalgae (separating cells from the culture medium) and for extracting lipids from the cell have proven to be energy-intensive and considerably affect biomass production's energy balance. This stage may, in fact, run as high as 50% of the production cost.

The waste-to-energy conversion of this biomass assumes that the entire cell is used (lipid extraction, thermochemical conversion, liquefaction, combustion, methanation). Moreover, fatty acid extraction generates nutrient-rich (in particular, nitrogen and phosphorus) waste material that requires recycling.

### PRESENTATION OF THE INVENTION

This invention targets a CO₂ fixation and organic waste processing method enabling the production of bioenergy in the form of methane, by combining the methanation waste processing technique with the gas purification technique using phytoplanktonic microorganisms such as microalgae and photosynthetic bacteria (such as cyanobacteria), in such a way as to optimize their respective technical advantages while entirely or partially eliminating the disadvantages of each technique when considered individually.

One goal of the invention is to provide a system for treating large quantities of gaseous effluents, specifically from industrial sources, by fixating the CO₂ contained therein.

To do this, the invention combines a solid organic waste methanation step with a step for filtering the biogas produced by photosynthetic microorganisms; said microorganisms are simultaneously supplied with inorganic carbon originating from a CO₂-containing gaseous effluent. In order to avoid or at least reduce the associated risks that the system of the invention might become inhibited through ammonium accumulation, particularly during the methanation step, the invention proposes that a high carbon/nitrogen (C/N) ratio be maintained inside the digester for the methanation step and/or inside the phytoplanktonic microorganisms' culture medium, if possible a ratio between 10 and 35, whereas this ratio normally ranges from 6 to 9, which then results in very high ammonium production and greatly inhibits the methanation step. Either one acts directly upon the C/N ratio in the digester, whose liquid phase must supply the methanizer in the methanation step, or one acts upon the microorganism culture unit, a fraction of which must be reintroduced into the digester, and therefore indirectly into the methanizer.

Since microorganisms are naturally programmed to grow, the invention uses part of said phytoplanktonic microorganisms to supply the digester for the methanation step. According to implementations of the method of the invention, it is possible to adjust the input of microorganisms and/or of organic waste, which may form a co-substrate, inside the digester. Hence, combining a processing method involving methanation with filtration using microorganisms creates an additional source of organic waste for methanation by using surplus biomass. By “surplus,” we mean part of the biomass present inside the microorganisms' culture tank that can be removed from said tank without negatively impacting the proper sequence of the biogas purification step of the method of the invention. To the extent that, conversely, increasing the biomass may quickly impede the performance of the microorganisms' culture tank, the expert will quickly find the correct balance between the quantity of biomass to be maintained inside the microorganisms' culture tank and the quantity of surplus biomass to remove in order to ensure optimal productivity, e.g., by maintaining turbidity such that 95% of the light is attenuated at a thickness of 50% to 90% of the depth of the pond.

Therefore, the method of the invention may use organic waste whose nature and quantity vary depending upon the phytoplanktonic microorganisms that are present inside the digester, in order to ensure optimal CO₂ fixation inside the phytoplanktonic microorganism culture unit. Similarly, the method of the invention takes into account the nature of the organic waste to be processed along with the composition of the microorganisms in order to adapt the processing method to the nature of said organic waste to be processed. This enables homogeneous processing of all types of organic waste.

Conversion of organic matter during methanation produces a biogas that contains a mixture of methane and CO₂, as well as a digestate whose liquid fraction contains organic acids and mineralized elements (such as nitrogen, phosphorus, etc.). The biogas, along with the digestate, will supply the microorganism culture unit.

The microorganism culture unit may be an open pond that makes it possible to solubilize a large quantity of CO₂. In order to limit contamination by microorganisms from outside the initial culture, it is possible to use species of extremophile microorganisms; that is, microorganisms that can survive in very high or very low pH environments. In particular, it is possible to use microorganisms such as Chlorella, Chloridella, Chlamydomonas, Viridiella, Euglena, and Euchromonoas for acid media and Arthrospira, Nannochlorposis, Synecococcus, and Tetraselmis for alkaline media, or a mixed population such as those found in treatment lagoons.

The culture unit advantageously has two independent CO₂ intake systems. One is for the CO₂ contained in the industrial-origin gaseous effluent and the other is for the CO₂ contained in the biogas to be purified. In the second system, fixation of the CO₂ contained in the biogas yields, once the process is completed, a gas with a high methane concentration and with increased energy potential.

Therefore, the goal of the invention is a CO₂ fixation and organic waste processing method that combines an anaerobic digestion system with a system for producing phytoplanktonic microorganisms, comprising the following steps:

(a′) microorganisms (105) originating from a phytoplanktonic culture and organic waste (104) are processed inside a hydrolysis reactor (101);

(a″) at least part of a liquid effluent (109) from Step (a′) is processed inside a methanation reactor (102);

(b) a liquid phase (127) and biogas to be purified (110) from Step (a″) is processed inside a phytoplanktonic microorganism culture unit (103);

(c) a gaseous effluent (113) containing CO₂ is injected into the phytoplanktonic microorganism culture unit;

(d) an NH₃ concentration less than 0.5 g/L is maintained inside the methanation reactor;

(e) a methane-enriched biogas is recovered when it exits the phytoplanktonic microorganism culture unit.

The biogas to be purified that is recovered after Step (a″) refers to a gas produced by the fermentation of organic matter, of animal and/or plant origin, in the absence of oxygen. The biogas is primarily composed of methane and carbon dioxide with, as applicable, small quantities of water, hydrogen sulfide, etc.

The methane-enriched, purified biogas recovered after Step (e) primarily contains methane and has a limited oxygen content. It is also possible for other gases to be present, such as: CO₂ and/or N₂, but in trace amounts. Generally speaking, the methane-enriched purified biogas recovered after Step (e) contains at least 90% methane.

Methanation in the two steps (a′) and (a″) makes it possible to place hydrolytic/acidogenic and methanogenic populations under their respective optimal conditions, to achieve higher conversion yields for the organic matter, and to process a broader spectrum of organic waste thanks to the buffering effect of the hydrolysis reactor. Additionally, the biogas produced in the methanation step has an especially high methane concentration, and the CO₂ produced during the hydrolysis step (a′) may advantageously be injected into the microalgae culture, in the same way as the biogas produced during the actual methanation step (a″).

Of course, it is possible, due to space constraints or for other reasons, to perform methanation in known fashion in a single step (a); the biogas to be purified is then recovered when it exits the single reactor.

The methanation step, due to organic acid consumption, tends to increase the pH inside the methanizer, or methanation reactor, resulting in an increase of the NH₃/NH₄⁺ ratio inside the methanizer. An overly-high NH₃ concentration inside the methanizer may run the risk, over the short term, of inhibiting the method of the invention.

Therefore, according to the invention, in order to maintain an NH₃ concentration under 0.5 g/L inside the methanizer, it is proposed that an average Carbon/Nitrogen (C/N) ratio of between 10 and 35 be maintained inside the hydrolysis reactor and/or inside the microorganism culture unit and/or that CO₂ be injected directly into the methanizer in order to limit the pH rise.

By “average C/N ratio,” we mean the average of the C/N ratios inside the waste, the cells, and inside the culture medium, as the microorganisms can excrete a large quantity of carbon in the form of organic polymers. Maintaining a C/N ratio ranging from 10 to 35, which is the consequence of a high CO₂ supply inside the phytoplanktonic microorganism culture unit, prevents the method of the invention from being inhibited due to ammonium accumulation.

To do this, according to the invention, one may, for example, adjust the quality and/or quantity of the fraction of organic waste brought into the hydrolysis reactor in Step (a′). Specifically, organic waste having a C/N ratio over 25 may be used.

Otherwise, it is possible to adjust the quantity of liquid effluent exiting Step (a″) and introduced in Step (b) in such a way as to induce a nutrient limitation that is able to modify the composition of the microorganisms inside the phytoplanktonic microorganism culture unit in order to encourage the accumulation of lipids and carbohydrates inside said microorganisms, and therefore to increase the C/N ratio inside said unit.

It is also possible to adjust the intake flow rate of the biogas exiting Step (a″) into the phytoplanktonic microorganism culture unit in order to control said culture unit's pH and to create the proper conditions for increasing the C/N ratio.

The quality of the intracellular contents of the microalgae depends, to a certain extent, upon their culture conditions. Hence, by altering the operating conditions applied while the microorganisms are cultured, it is possible to modify considerably the distribution of protein, lipid, and carbohydrate compartments in the organic matter. These modifications influence the cells' biodegradability and their conversion into organic acids and methane. The variations between the nitrogenous (proteins) and carbonaceous (carbohydrates and lipids) fractions of the organic matter affect the overall carbon/nitrogen (C/N) ratio inside the hydrolysis reactor and therefore inside the phytoplanktonic microorganism culture unit as well.

According to the invention, controlling the microorganisms' culture conditions can therefore be performed by adjusting the CO₂ intake and/or by adjusting the intake of nutrients contained in the liquid phase recovered after steps (a′) and/or (a″). In any case, the CO₂ originates from a gaseous effluent with a high CO₂ content (from 5 to 25%) but may also originate from the hydrolysis reactor, where the first organic waste processing step occurs. More specifically, according to the invention, it is possible to incorporate a step wherein one adjusts the quantity of liquid effluent exiting from Step (a″) that is introduced into the phytoplanktonic microorganism culture unit, and/or the quantity of organic and/or inorganic CO₂ introduced into said microorganism culture unit, in such a way as to modify the composition of the microorganism culture unit's biomass and to maintain a C/N ratio of between 10 and 35.

Controlling these two parameters (nutrients and CO₂) makes it possible to modify the composition of the cells, and hence the C/N ratio. The microorganisms' rapid growth (roughly a doubling of the population per day) allows for rapid cellular response.

Under other conditions, the qualities and quantities of co-wastes may be adjusted so that they conform to the quality of the microorganisms in order to remain under optimal breakdown conditions.

By increasing the intake flow of the liquid phase into the microorganism culture unit, this step encourages biomass growth, since the intake of nutrients and nitrogen is increased. This liquid phase contains, along with organic acids, primarily nitrogen and phosphorus in mineral form (ammonium and phosphate). The latter elements are necessary for the metabolism of phytoplanktonic microorganisms such as microalgae. Certain organic acids can be assimilated by certain microalgae species (heterotrophic or mixotrophic growth) and significantly increase growth.

Conversely, by decreasing the intake flow of the liquid phase into the microorganism culture unit, the C/N ratio is increased inside said culture unit. Nutritional deficiencies impact the physiology of microalgae cells. Nitrogen limitation or deficiency encourages carbon accumulation inside the cell in the form of carbohydrates, starch, or lipids. However, this phenomenon is accompanied by slower growth, since the nitrogen precursor needed for protein development is not present. Therefore, it is necessary to control the intake flow of liquid effluent such that it does not result in a total stoppage that would harm microorganism growth.

Likewise, by adjusting the biogas flow rate exiting from Step (a″) injected into the microorganism culture unit, one may modify the composition of the biomass in the phytoplanktonic microorganism culture unit (but also their productivity) depending upon how much carbon and nitrogen are needed. By increasing the quantity of biogas in the phytoplanktonic microorganism culture unit, the accumulation of starch and lipids inside the cells is encouraged, which makes it possible to increase the C/N ratio in the microorganisms.

Similarly, the intake flow of the CO₂-containing gaseous effluent may be adjusted, since the CO₂ supply modifies the composition of the biomass in the microorganism culture unit. If the culture has an alkaline pH, the CO₂ will dissolve spontaneously. Regardless of whether the culture is acid or alkaline, the addition of gas will be determined by pH via a regulation system. The injection of CO₂-rich gas into the medium is advantageously linked to the pH such that the pH is maintained at a set point. PID- or MLI-type regulators may be used for this purpose. If alkalinity is high (large quantities of cations are present), the pH can be maintained at high values despite the CO₂ flows.

The gaseous effluent intake flow can also be adjusted so as to maintain a set carbon flow that is at least 10 times higher than the nitrogen flow entering the microorganism culture unit. To do this, one may establish a dilution rate for the phytoplanktonic microorganism culture that is lower than the maximum growth rate of said microorganisms, so as to induce a nutrient limitation, since a surplus of inorganic carbon is being supplied to it.

Moreover, adding CO₂, preferably linked to the pH value, to the microorganism culture unit makes it possible to maintain the dissolved CO₂ within a range that does not limit photosynthesis. This prevents an often-encountered phenomenon wherein the pH rises in high-density cultures, associated with a depletion of the dissolved inorganic carbon that is needed for growth. Therefore, injecting CO₂, for acidophilic cultures and alkaline cultures, makes it possible to maintain high growth rates, including for high biomass concentrations inside the culture, by maintaining a slightly acid pH (between 4 and 7). Only certain microalgae are able to develop within these pH ranges, which limits biodiversity; as has been observed in natural ecosystems, the latter then becomes simpler in its composition.

The addition of CO₂ also significantly increases biomass concentration, which leads to an increase in the purifying capacities of nitrogen and phosphorus.

It is also possible, in order to maintain an average C/N ratio of between 10 and 35 inside the phytoplanktonic microorganism culture unit, to use organic waste that has a C/N ratio over 25.

Similarly it is possible to use autotrophic species, which only consume inorganic carbon, with a C/N ratio over 10, as microorganisms for Step (b) (e.g., Staurastrum sp.).

All or part of these solutions for maintaining an average C/N ratio of between 10 and 35 inside the hydrolysis reactor and inside the microorganism culture unit may be used concurrently in order to maintain said ratio in the desired proportions and therefore to maintain an NH₃ concentration under 0.5 g/L inside the methanizer.

Otherwise, as was mentioned earlier, in order to maintain an NH₃ concentration under 0.5 g/L inside the methanizer, an additional step (f) may be used, during which a CO₂-containing gaseous effluent is introduced into the methanation reactor of Step (a″) in order to maintain therein a pH of roughly 7.5, and more generally ranging from 7 to 8, in order to prevent methanogenesis inhibition due to ammonia accumulation. The main effect of ammonium production is alkalinization of the medium, which causes pH to rise, thereby encouraging the (toxic) NH3 form to the detriment of the NH4⁺ form. The injection of CO₂ into the digester, using an ad hoc pump, is linked to the methanation reactor's pH such that the pH is maintained at about 7.5. It should be noted that this “natural” alkalinity production effect eliminates the need for alkaline chemicals such as sodium hydroxide or potassium bicarbonate, which increase costs for remediation devices.

Moreover it is possible, when the pH exiting Step a′ is naturally alkaline, according to an extra step (g), to filter the biogas from Step (a″) on an [ion] exchange column wherein the biogas is injected from below. When the bubbles rise, the CO₂ dissolves and turns into bicarbonate, whereas the low-solubility methane is recovered at the reactor's surface prior to Step (b), so as to take advantage of the alkalinity induced by the ammonium in order to dissolve the CO₂ and to recover the purified methane. This also results in limiting basification of the medium inside the methanation reactor, therefore lowering the ammonium's toxicity. The biogas purified during this intermediary step (f) still contains some CO₂ and is therefore advantageously injected into the phytoplanktonic microorganism culture unit so that it may be fully fixated therein.

It is also possible, according to the method of the invention, to inject a fraction of the liquid effluent exiting Step (a′) directly into the phytoplanktonic microorganism culture unit. This type of injection preferably occurs at night so that it does not compete with photosynthesis and so that microalgae biomass production is ensured during the nocturnal phase.

Advantageously, acidophilic or basophilic species are used inside the phytoplanktonic microorganism culture unit in order to limit contaminations within said unit.

Advantageously, the biogas recovered following Step (a″) is temporarily stored inside a buffer tank before it is introduced into the microorganism culture unit. Using this type of tank makes it possible to easily adjust the supply of biogas inside the microorganism culture tank as needed and to store the biogas during the nocturnal phase.

The method of the invention also makes it possible to produce another biofuel in addition to the methane-enriched purified biogas, by extracting and recovering recoverable compounds, such as lipids, from the microorganism biomass, prior to injecting the residue—that is, the biomass that is free of recoverable compounds—into the hydrolysis reactor.

Preferably, the biomass originating from the microorganism culture unit is concentrated such that the supernatant is separated from said biomass, prior to introducing the biomass concentrate into the hydrolysis reactor; the liquid phase may be reintroduced into the microorganism culture unit.

The invention also relates to a combined organic waste processing and CO₂ fixation system that implements the method of the invention, comprising at least
a hydrolysis/acidogenesis reactor connected to a methanation reactor,
a phytoplanktonic microorganism culture unit,
a first supply pipe for bringing a biogas to be purified from the methanation reactor to the phytoplanktonic microorganism culture unit,
a second supply pipe for bringing a nutrient-rich liquid phase from the hydrolysis reactor and/or the methanation reactor to the phytoplanktonic microorganism culture unit,
a third supply pipe for bringing a CO₂-containing gaseous effluent from outside said system to the phytoplanktonic microorganism culture unit, and
a pipe for discharging and recovering the methane-enriched purified biogas after it exits the phytoplanktonic microorganism culture unit.

### DETAILED DESCRIPTION OF THE FIGURES AND OF THE INVENTION

The invention will be more fully understood by reading the following description and by referring to the accompanying drawings. These are presented for informational purposes and in no way limit the invention. The figures show:

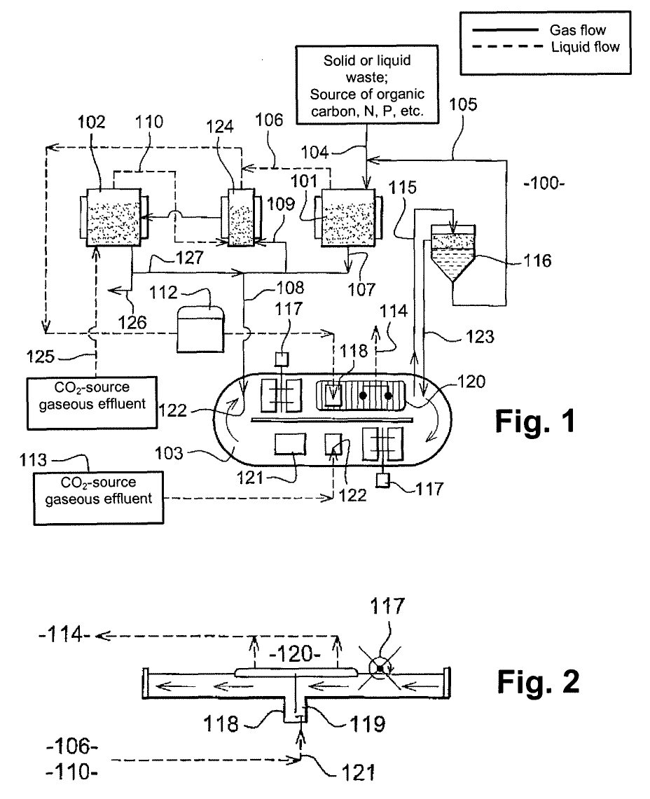

FIG. 1: a schematic representation of an installation according to one embodiment implementing the method of the invention;

FIG. 2: an enlarged view of the device for purifying the biogas contained inside the phytoplanktonic microorganism culture unit in FIG. 1.

In the example shown in FIG. 1, an organic waste processing and CO₂ fixation system 100 implementing the method of the invention comprises three principal units: respectively, the hydrolysis/acidogenesis reactor 101, the methanation reactor 102, and the phytoplanktonic microorganism culture unit 103.

The phytoplanktonic microorganism culture unit 103 is, for example, a shallow pond (20 to 50 cm). Stirring is performed in known fashion by two paddle wheels 117 that recirculate and mix the culture medium and the microorganisms.

The organic matter originating from organic waste 104, on the one hand, and from the microorganism cultures and/or from microorganism residue 105, following optional extraction of recoverable compounds, on the other hand, is injected via supply pipes into the hydrolysis/acidogenesis reactor 101, inside which hydrolytic and acidogenic organisms (Clostridium Bacillus Escherichia Staphylococcus, etc.) convert it into organic acids and hydrolyzed molecules.

Elements contained inside the organic matter, such as nitrogen and phosphorus, are also mineralized.

The produced gas 106 is primarily composed of carbon dioxide and, to a lesser extent, hydrogen.

A fraction 108 of the liquid output 107 from the hydrolysis/acidogenesis reactor 101, whose pH is generally acidic (around 5 or 6), is introduced via a specific supply pipe into the phytoplanktonic microorganism culture unit 103. This increases biomass production, encourages biomass growth in the absence of light (therefore, at night), and enables lipid accumulation under nitrogen-deficient conditions.

The remainder 109 of the liquid output 107 is introduced into a biogas filtration reactor 124 before being introduced into the methanation reactor 102. This filtration reactor 124 is used when the low C/N ratio (between 5 and 20) upon entering the hydrolysis/acidogenesis reactor 101 leads to a large release of ammonium and therefore to pH levels over 8. The alkalinity of the liquid output 107 is then used to dissolve the CO₂, whereas the low-solubility methane is released out of the top of the methanizer.

The methanogenic population (methanosarcina, methanococcus, methanobacterium, etc.) of the anaerobic methanation reactor 102 converts these acids into a biogas 110 that is primarily composed of methane and carbon dioxide.

Advantageously, the biogas 110 also passes through the filtration reactor 124 before being injected via a supply pipe into the phytoplanktonic microorganism culture unit 103. This is therefore the initial biogas filtration. The ammonium-induced alkalinity makes it possible to dissolve part of the CO₂ contained in the biogas to be purified and to recover the partially-purified methane. This also limits basification of the medium inside the methanation reactor 102 and therefore lowers the ammonium's toxicity.

Part of the digestate 126, or the liquid phase, resulting from the methanation stage may also be used for agricultural recovery after it leaves the methanation reactor 102. The remainder 127 is introduced into the-microorganism culture unit 103, along with the liquid effluent fraction 108 from the hydrolysis reactor 101.

The microorganisms present inside the culture unit 103 use, for their nutritional needs, elements such as NH₄⁺ and PO₄³⁻ originating from the hydrolysis/acidogenesis reactor 101 and, as a carbon source, the CO₂ present in the gases 106, 110 that are recovered after they exit the hydrolysis/acidogenesis 101 and methanation 102 reactors and that travel through supply pipes to the microorganism culture unit 103. These gases 106, 110 are advantageously stored inside a tank 112 before being injected into the microorganism culture unit 103. The gas 106, 110 originating from the hydrolysis 101 and methanation 102 reactors contains, along with methane, CO₂, but may also contain sulfur and/or other compounds that may negatively impact direct use of the biogas, and must therefore be purified.

More specifically, the gases 106, 110 originating from the hydrolysis/acidogenesis 101 and methanation 102 reactors are injected into the pond 121 of the microorganism culture unit 103 using a purification device 118 (see enlarged view in FIG. 2) known to the expert, which encourages both transfer of the gas flow 119 and recovery of the gas inside a recovery cap 120 after passing inside the culture unit 103.

The purification device 118 comprises a pond 121 equipped with several wells 100 to 150 cm deep, at the bottom of which diffusers are located. Transfer occurs via these wells, which generate very small gas bubbles, ensuring optimal transfer to the liquid phase, and the culture medium passes through this column of bubbles. When the microorganism culture arrives at the diffusers, it has consumed most of the dissolved inorganic carbon stock, and the pH has risen to higher values. The CO₂ contained in the gas is therefore quickly transferred into the culture medium, where it is stored mainly in the form of bicarbonate. The non-fixated CO₂, as well as the gas compounds (namely methane and hydrogen) that are not transferred to the liquid phase are recovered on the surface of the pond 121, while ensuring minor depressurization as needed.

The ongoing CO₂ intake makes it possible to maintain, at certain points in the pond 121, during the day, a pH set to an acid (under 6.5) or basic (over 8.5) value, thereby limiting the development of undesirable microorganisms.

Solubilizing CO₂ (in the form of bicarbonate and CO₂) involves controlling the pH at the gas injection points 122 in order to ensure that the inorganic carbon for the microorganisms is never restricted (pH under 8.5 for nonalkaliphilic species), that the medium at the gas injection point 122 has a low inorganic carbon concentration for rapid transfer of the CO₂ to the liquid phase, whereas the methane remains primarily in gas form since its solubility is much lower, and in order to maintain local acidic conditions.

Moreover, since the gas injection point 122 is located near the paddle wheels 117, the dissolved oxygen is massively degassed, which considerably limits its presence in the recovered biogas whose CO₂ has been purified. Consequently, at the injection point 122, the pH value of the medium is higher, since the CO₂ has been consumed, and the dissolved oxygen concentration is very low.

The pH set points at the injection points 122 are calculated to ensure that the culture medium arriving at the injection point 122 has sufficient CO₂ storage capacity. In this way, most of the CO₂ is absorbed, whereas the methane passes through the liquid phase and becomes much more concentrated inside the gas (see pH calculation example below).

The filtration operation for the biogas to be purified 110 by the phytoplanktonic microorganisms in the culture unit 103 produces at the outlet 114 a purified biogas with a high methane content, which can be converted into energy or stored for future use.

A gaseous effluent 113, originating, for example, from human activity, is also used as a carbon source for supplying the microalgae culture unit 103. This gaseous effluent 113 travels through a supply pipe from the source, e.g., a tank, to the phytoplanktonic microorganism culture unit.

The same filtration device 118 is used for fixating the CO₂ contained in the gaseous effluent 113, except that the latter does not necessarily have the gas recovery cap 120.

The addition of the CO₂-source gaseous effluent 113 is conditioned by the pH via a regulation system. The CO₂-rich gas flow is regulated depending upon the pH downstream of the injection point so as to maintain the pH at a set point. A PID-, RST- or MLI-type regulator is advantageously used. The distance between the pH probe and the gas injection point must correspond to the distance traveled by the moving fluid between two moments of injection. For example, if the velocity of the fluid is 30 cm/sec., and if calculation of the injection by the regulation system is launched every 3 seconds, the probe must be located 90 cm from the injection point.

The surplus algal and bacterial biomass 115 is removed from the microalgae culture unit 103. To improve the system's efficiency, the biomass removal is managed so as to ensure near-optimal system productivity (which varies depending upon incident light); operations using a turbidostat makes it possible to control the concentration depending upon incident light and to ensure that the culture is consistently under limited-nutrient conditions. The surplus biomass 115 is directed towards a decanter 116 and the decantate 105, whose recoverable compounds have been extracted, if desired, travels through a pipe to the hydrolysis/acidogenesis reactor 101.

Depending upon how the processing system 100 is used, the decantate 105 may be used as the sole substrate inside the hydrolysis/acidogenesis reactor 101, or in a mixture in order to be codigested along with organic waste 104. The supernatant 123 is, as needed, reused in the microalgae culture unit 103.

An exterior CO₂ source 125, in the form of a CO₂-containing gaseous effluent, e.g., from an industrial source, may be introduced into the methanation reactor 102 in order to maintain a pH of around 7.5 therein. The gas 110 exiting the methanation reactor 102 is then heavily loaded with inorganic carbon.

Quality control for the biomass in the phytoplanktonic microorganism culture unit 103 improves quality by increasing the intracellular lipid or starch concentration as needed; that is, depending upon the C/N ratio to be maintained between 10 and 35, but also if one wishes to optimize organic waste processing based on the nature of the organic waste 104 to be processed inside the hydrolysis reactor 101. This also increases the quantity of purified biogas 114 produced per mass unit of microorganisms.

Before being injected into the hydrolysis reactor 101, the microorganisms 105 may undergo physical and/or chemical pre-processing (thermal, acid/base, ozonation, etc.) in order to improve their digestibility and to thereby increase their productivity.

The recycled biomass 105 may be combined with another substrate, with or without pre-processing.

### Examples

### 1.1 Example for Calculating pHs to be Used Inside the Microorganism Culture Unit

The example is given for a culture in an open pond 150 m long whose instant CO₂ fixation rate is 2 mmol/l/hr. These are the traditional average values for this type of microorganism culture unit, corresponding, for example, to a biomass of 0.4 g/l and an instant growth rate of 2.3 day⁻¹.

This induces pH increases, depending upon the medium's alkalinity, of 0.1 to 0.5 pH units per minute.

For a culture flowing at 30 cm·s⁻¹, the 75 meters between the two diffusers are reached in 4.2 minutes, which corresponds to a pH increase from 0.4 to 2.1 pH units. This pH increase is reinforced after passing through the paddle wheels, which degasses part of the CO₂.

The pH set point at the gas injection point must then be set at a value between 5 and 6.5. For a control loop launched every 3 seconds, the pH probe must be placed 90 cm downstream of the injection point.

### 1.2 Dimensioning Example

Since the respective maximum dilution rates for the anaerobic digestion method and for the phototrophic culture are of the same order of magnitude, the ratio of the volumes between the pond where the microorganisms are cultured and the methanizer will correspond to the concentration factor of the microorganisms after they are sampled. For example, if the microorganisms have been concentrated by a factor of 100, the associated flow rate to be processed will be 100 times lower, and the methanizer's volume will therefore be 100 times lower. Hence, one may envision going from 0.5 g/l of dry matter in the pond to 50 g/l in the methanizer with a flow rate that is 100 times lower. The input value of 50 g/l for the methanizer corresponds to a value of 25 g/l of carbon, or 1 to 4 g/l of nitrogen, which is therefore a maximum level not to be exceeded.

For a pond depth of 10 cm, this corresponds to a 1000 m³ digester per hectare of pond.

The pond will then have a processing capacity of 0.5 g/l/day, or 0.25 g C/l/day, or 0.9 CO₂/l/day, or 900 kg of CO₂ per hectare. The production associated with 500 kg of dry matter per hectare per day is 600 kg of DCO per hectare, which corresponds to 220 m³ of methane (and 95 m³ of CO₂, or 185 kilos of CO₂). This corresponds to an energy content of 1800 kWh. Additionally, the nitrogen flow per day per hectare is 10 to 40 kilograms, which would have had to be provided using fertilizer if the nitrogen had not been recycled via anaerobic digestion. The input from the methanation step is on the order of 370 kilograms of inorganic carbon.

### 1.3 Implementation Example of the Method

Based on the preceding calculations, for a one-hectare pond:
nature of organic waste and quantities: distillery residue; 1 m³/day at 50 kg/m³ (C/N=200).
quantities of liquid effluent 108 and 127 introduced into the microorganism culture unit 103: respectively, 0 m³/day and 9 m³/day
nature of microorganisms used: chlamydomonas acidophila-type acidophilic microalgae maintained at pH 4 (C/N=6)
quantity 105 of microorganisms sampled inside the culture unit and introduced into the hydrolysis reactor 101: 500 kg/day, or 9 m³ and 104 1 m³/day, or 50 kg, with the average C/N ratio then being 25.
pH at the injection points 122 inside the pond 118: pH 4
quantity of CO₂-containing industrial effluent 113 introduced into the microorganism culture unit 103: 715 kg of fixated CO₂, which corresponds to an injection (for a 20% transfer efficiency) of 3.5 tons of industrial CO₂.
quantity of CO₂-containing industrial effluent 125 introduced into the methanation reactor 102: 0, since C/N ratio maintained between 10 and 35 inside the microalgae culture unit.
quantity of methane-enriched purified biogas recovered upon exiting the microorganism culture unit: 220 m³/day
operating time for the system: System is cleaned every 6 months.

### BIBLIOGRAPHY

Angelidaki I, Ahring B K. Thermophilic anaerobic digestion of livestock waste: the effect of ammonia. Appl. Microbiol. Biotechnol. 1993;38:560-564.

Angelidaki I, Ellegaard L, Ahring B K. Applications of the anaerobic digestion process. In: Ahring B K, editor. Biomethanation, Springer Berlin/Heidelberg, 2003. p. 1-33.

Braun R, Huber P, Meyrath J. Ammonia toxicity in liquid piggery manure digestion. Biotechnol. Lett. 1981;3:159-164,

Chen P H. Factors influencing methane fermentation of micro-algae. PhD thesis, University of California, Berkeley, Calif., USA, 1987.

Chen Y, Cheng J J, Creamer K S. Inhibition of anaerobic digestion process: A review. Bioresour. Technol. 2008;99:4044-4064.

Chisti Y. Biodiesel from miocroalgae. Biotechnol. Adv. 2007;25: 294-306.

Cirne D G, Paloumet X, Björnsson L, Alves M M, Mattiasson B. Anaerobic digestion of lipid-rich waste—Effects of lipid concentration. Renew. Energy. 2007;32:965-975.

Doucha J, Straka F, Livansky K. Utilization of flue gas for cultivation of microalgae (Chlorella sp.) in an outdoor open thin-layer photobioreactor. J. Appl. Phycol. 2005:17:403-412,

Golueke C G, Oswald W J, Gotaas H B. Anaerobic digestion of algae. Appl. Microbiol. 1957;5:47-55.

Grobbelaar J U. Algal Nutrition. In Richmond A, editor. Handbook of microalgal culture: biotechnology and applied phycology. Wiley-Blackwell, 1984.

Heubeck 5, Craggs R J, Stilton A. Influence of CO₂ scrubbing from biogas on the treatment performance of a high rate algal pond. Water Sci. Technol. 2007;55(11):193-200.

Koster I W, Lettinga G. The influence of ammonium-nitrogen on the specific activity of pellitized methanogenic sludge. Agric. Wastes. 1984;9:205-216.

Koster I W, Lettinga G. Anaerobic digestion at extreme ammonia concentrations. Biol. Wastes 1988;25:51-59.

Maeda K, Owada M, Kimura N, Omata K, Karube I. CO₂ fixation from me flue gas on coal-fired thermal power plant by microalgae. Energy Conv. Manag. 1995.36:717-720.

Mendeno G, Craggs R, Tanner C, Suskias J, Webster-Brown J. Potential biogas scrubbing using a high rate pond. Water Sci. Technol. 2005;51(12)253-256.

Munoz R, Jacinto M, Guleysse B, Mattiasson B. Combined carbon and nitrogen removal from acetonitrile using algal-bacterial bioreactors. Appl. Microbial. Biotechnol, 2005;67:699-707.

Olaizola M. Commercial development of microalgal biotechnology: from the test tube to the marketplace, Biomol. Eng. 2003;20:459-466.

Omil F, Mendez R, Lema J M. Anaerobic treatment of saline wastewaters under high sulfide and ammonia content. Bioresour. Technol. 1995;54:269-278.

Oswald W J, Golueke C C. Biological transformation of solar energy. Adv. Appl. Microbiol. 1960;2:223-262.

Samson R, LeDuy A. Biogas production from anaerobic digestion of Spirulina maxima algal biomass. Biotechnol. Bioeng. 1982;24:1919-1924.

Samson R, LeDuy A. Detailed study of anaerobic digestion of Spirulina maxima algae biomass. Biotechnol. Bioeng. 1986;28:1014-1023.

Sanchez E P, Travieso L. Anaerobic digestion of Chlorella vulgaris for energy production,” Resour. Conserv. Recycl. 1993;9:127-132.

Travieso L, Sanchez E P, Benitez F, Conde J L. Arthospira sp. intensive cultures for food and biogas purification. Biotechnol. Lett. 1993;15:1091-1094.

Yen H W, Brune D E. Anaerobic co-digestion of algal sludge and waste paper to produce methane. Bioresour. Technol. 2007;98:130-134.

1. A method for CO₂ fixation and treatment of organic waste by combining an anaerobic digestion system with a system for producing phytoplanktonic microorganisms, comprising the following steps:
(a′) microorganisms originating from a phytoplanktonic culture and organic waste are processed inside a hydrolysis reactor;
(a″) at least part of a liquid effluent from Step (a′) is processed inside a methanation reactor;
(b) a liquid phase and biogas to be purified from Step (a″) is processed inside a phytoplanktonic microorganism culture unit;
(c) a gaseous effluent containing CO₂ is injected into the phytoplanktonic microorganism culture unit;
(d) an NH₃ concentration under 0.5 g/L is maintained inside the methanation reactor; and
(e) a methane-enriched biogas is recovered when it exits the phytoplanktonic microorganism culture unit.

2. The CO₂ fixation and organic waste treatment method of claim 1, wherein, in order to maintain an NH₃ concentration under 0.5 g/L inside the methanation reactor, the following additional step is used:
(f) a CO₂-containing gaseous effluent is injected into the methanation reactor.

3. The CO₂ fixation and organic waste treatment method of claim 1, wherein, in order to maintain an NH₃ concentration under 0.5 g/L inside the methanation reactor, an average carbon/nitrogen (C/N) ratio between 10 and 35 is maintained inside the hydrolysis reactor.

4. The CO₂ fixation and organic waste treatment method of claim 3, wherein, in order to maintain an average C/N ratio between 10 and 35 inside the hydrolysis reactor, the fraction of organic waste placed inside the hydrolysis reactor in Step (a′) is adjusted.

5. The CO₂ fixation and organic waste treatment method of claim 3, wherein, in order to maintain an average C/N ratio between 10 and 35 inside the hydrolysis reactor, organic waste having a C/N ratio over 25 is used inside the hydrolysis reactor.

6. The CO₂ fixation and organic waste treatment method of claim 1, wherein, in order to maintain an NH₃ concentration under 0.5 g/L inside the methanation reactor, an average carbon/nitrogen (C/N) ratio between 10 and 35 is maintained inside the phytoplanktonic microorganism culture unit.

7. The CO₂ fixation and organic waste treatment method of claim 6, wherein, in order to maintain an average C/N ratio between 10 and 35 inside the phytoplanktonic microorganism culture unit, autotrophic species having a C/N ratio over 10 are used as microorganisms for Step (b).

8. The CO₂ fixation and organic waste treatment method of claim 6, wherein, in order to maintain an average C/N ratio between 10 and 35 inside the phytoplanktonic microorganism culture unit, the quantity of liquid effluent exiting Step (a″) and entering Step (b) is adjusted in such a way as to induce a nutrient limitation that is able to modify the composition of the microorganisms inside the phytoplanktonic microorganism culture unit in order to encourage the accumulation of lipids and carbohydrates inside said microorganisms.

9. The CO₂ fixation and organic waste treatment method of claim 6, wherein, in order to maintain an average C/N ratio between 10 and 35 inside the phytoplanktonic microorganism culture unit, the intake flow rate of the biogas exiting Step (a″) into the phytoplanktonic microorganism culture unit is adjusted in order to control the pH of the culture unit and to create the proper conditions for increasing the C/N ratio.

10. The CO₂ fixation and organic waste treatment method of claim 6, wherein, in order to maintain an average C/N ratio between 10 and 35 inside the phytoplanktonic microorganism culture unit, the CO₂-containing gaseous effluent intake flow from Step (c) into the microorganism culture unit is adjusted so as to maintain a carbon flow that is at least 10 times higher than the nitrogen flow.

11. The CO₂ fixation and organic waste treatment method of claim 10, wherein a dilution rate is established for the phytoplanktonic microorganism culture that is lower than the maximum growth rate of microorganisms of the culture, so as to induce a nutrient limitation.

12. The CO₂ fixation and organic waste treatment method of claim 1, wherein acidophilic or basophilic species are used inside the phytoplanktonic microorganism culture unit in order to limit contaminations within the unit.

13. The CO₂ fixation and organic waste treatment method of claim 1, further comprising the following step:
(g) the methane-enriched biogas from Step (a″) is filtered on an exchange column prior to Step (b).

14. The CO₂ fixation and organic waste treatment method of claim 1, further comprising the following step:
(h) a fraction of the liquid effluent exiting Step (a′) is injected directly into the phytoplanktonic microorganism culture unit.

15. A combined CO₂ fixation and organic waste treatment system comprising a hydrolysis/acidogenesis reactor connected to a methanation reactor, and a phytoplanktonic microorganism culture unit, a first supply pipe for bringing a biogas to be purified from the methanation reactor to the phytoplanktonic microorganism culture unit, a second supply pipe for bringing a nutrient-rich liquid phase from the hydrolysis reactor and/or the methanation reactor to the phytoplanktonic microorganism culture unit, a third supply pipe for bringing a CO₂-containing gaseous effluent from outside the system to the phytoplanktonic microorganism culture unit, and a pipe for discharging and recovering the methane-enriched purified biogas after it exits the phytoplanktonic microorganism culture unit.

16. The CO₂ fixation and organic waste treatment method of claim 1 further comprising the steps:
(f) injecting a CO₂-containing gaseous into the methanation reactor;
(g) filtering the methane-enriched biogas from Step (a″) on an ion exchange column prior to Step (b); and
(h) directly injecting a fraction of the liquid effluent exiting Step (a′) into the phytoplanktonic microorganism culture unit.

17. The CO₂ fixation and organic waste treatment method of claim 16, wherein, in order to maintain an NH₃ concentration under 0.5 g/L inside the methanation reactor, an average carbon/nitrogen (C/N) ratio between 10 and 35 is maintained inside the hydrolysis reactor.

18. The CO₂ fixation and organic waste treatment method of claim 17, wherein, in order to maintain an average C/N ratio between 10 and 35 inside the hydrolysis reactor:
(1) the fraction of organic waste placed inside the hydrolysis reactor in Step (a′) is adjusted; or
(2) organic waste having a C/N ratio over 25 is used inside the hydrolysis reactor.

19. The CO₂ fixation and organic waste treatment method of claim 17, wherein, in order to maintain an NH₃ concentration under 0.5 g/L inside the methanation reactor, an average carbon/nitrogen (C/N) ratio between 10 and 35 is maintained inside the phytoplanktonic microorganism culture unit.

20. The CO₂ fixation and organic waste treatment method of claim 19, wherein, in order to maintain an average C/N ratio between 10 and 35 inside the phytoplanktonic microorganism culture unit:
(1) autotrophic species having a C/N ratio over 10 are used as microorganisms for Step (b);
(2) the quantity of liquid effluent exiting Step (a″) and entering Step (b) is adjusted in such a way as to induce a nutrient limitation that is able to modify the composition of the microorganisms inside the phytoplanktonic microorganism culture unit in order to encourage the accumulation of lipids and carbohydrates inside said microorganisms;
(3) the intake flow rate of the biogas exiting Step (a″) into the phytoplanktonic microorganism culture unit is adjusted in order to control the pH of the culture unit and to create the proper conditions for increasing the C/N ratio; or
(4) the CO₂-containing gaseous effluent intake flow from Step (c) into the microorganism culture unit is adjusted so as to maintain a carbon flow that is at least 10 times higher than the nitrogen flow.
